(19) United States
(12) Patent Application Publication
(10) Pub. No.: US 2013/0059069 A1
(43) Pub. Date: Mar. 7, 2013

(54) METHOD FOR TESTING GLASS MATERIAL, METHOD FOR EVALUATING GLASS MATERIAL AND METHOD FOR PRODUCING OPTICAL ELEMENT

(71) Applicants: Shigeru Aoyagi, Shinjuku-ku (JP); Jun Nakamura, Shinjuku-ku (JP); Kazuo Tachiwana, Shinjuku-ku (JP); Junko Akojima, Shinjuku-ku (JP)

(72) Inventors: Shigeru Aoyagi, Shinjuku-ku (JP); Jun Nakamura, Shinjuku-ku (JP); Kazuo Tachiwana, Shinjuku-ku (JP); Junko Akojima, Shinjuku-ku (JP)

(73) Assignee: HOYA CORPORATION, Tokyo (JP)

(21) Appl. No.: 13/605,175

(22) Filed: Sep. 6, 2012

(30) Foreign Application Priority Data
Sep. 7, 2011 (JP) 2011-194832

(51) Int. Cl. B05D 5/06; G01N 33/00

(52) U.S. Cl. 427/165; 436/163

(57) ABSTRACT

There is provided a method for testing a glass material and a method for evaluating a glass material capable of preventing a generation of a gap between an evaluation result and an actual degradation state of the surface of a lens, and also to provide a method for producing an optical element capable of sufficiently exhibiting chemical durability possessed by the optical element, which is the method for testing a glass material, to perform a test regarding a chemical durability of a glass material, comprising examining a correlation between a variation of hydrogen ion concentration indexes of a process liquid in which the glass material is immersed, and a variation of chemical durability index values of a prescribed type of the glass material.

### BACKGROUND

1. Technical Field

The present invention relates to a method for testing a glass material, a method for evaluating a glass material and a method for producing an optical element, regarding a glass material being a material of forming an optical element.

2. Description of Related Art

A surface degradation (such as white fogging, blue fogging, and latent flaw) that can be generated in a glass material for forming an optical element such as a lens and a prism, is closely related to a chemical durability of the glass material. The chemical durability of the glass material means the durability when chemical reaction occurs between a glass material component and process liquid (cleaning liquid and polishing liquid, etc.), and more specifically water resistance, acid resistance, and detergent resistance, etc., can be given as the durability. Such a chemical durability is different depending on a chemical composition of the glass material, and therefore is indispensable for evaluating the glass material.

Conventionally, the chemical durability of the glass material is tested and evaluated by a technique defined by Japan Optical Glass Industry Association Standard. More specifically, for example a water resistance class powder method (Dw), acid resistance class powder method (D_{A}), blue fogging resistance surface method (T_{blue}), latent flow resistance (D_{NaoH}), latent flaw resistance (D_{STTP}), and chemical durability (D_{O}), are tested and evaluated, so that each type of these indexes is classified into grades 1 to 6 (for example, see non-patent documents 1 and 2).
Non-patent document 1: “Method for measuring chemical durability of optical glass (surface method)” by Japan Optical Glass Industry Association Standard JOGIS07-1975
Non-patent document 2: “Technical information (chemical property)”, [online], HOYA GROUP optics division, [searched in Sep. 7, 2011], Internet <URL:http://www.hoya-opticalworld.com/japanese/technical/003.html>

However, the above-described conventional technique sometimes involves a problem as described below, regarding the test and evaluation of the glass material.

For example, according to conventional evaluation result information, when six types (D_{W}, D_{A}, T_{blue}, D_{NaOH}, D_{STTP}, D_{O}) of the glass material are tested and evaluated, it is found that the chemical durability of all glass materials of glass type called “FDS18 (by HOYA Corporation)” is classified into grade 1, and these glass materials have excellent chemical durability. However, when a lens being an optical element is fabricated through a grinding step, a polishing step, and a cleaning step using cleaning liquid applied to the FDS18, there is a problem that a latent flaw is generated on a surface of the lens in a process of coating the surface of the lens with an antireflection film.

This means that an actual state of the surface of the lens is degraded with elapse of time, although the chemical durability of all glass materials is classified into grade 1, according to the evaluation of a conventional technique. This also means that there is sometimes a gap between an evaluation result by the conventional technique and an actual degradation state of the surface of the lens.

Therefore, an object of the present invention is to provide a method for testing a glass material and a method for evaluating a glass material capable of preventing a generation of a gap between an evaluation result and an actual surface state of the lens, and is also to provide a method for producing an optical element capable of sufficiently exhibiting a chemical durability possessed by the optical element.

### SUMMARY OF THE INVENTION

In order to achieve the above-described objects, inventors of the present invention study on a reason for degrading an actual state of the surface of the lens with elapse of time, irrespective of using the glass materials all of which having grade 1 chemical durability in an evaluation of a conventional technique.

As a result of such a study, it is assumed by the inventors of the present invention as follows, as the reason for degrading the actual surface state of the lens with elapse of time.

(Reason 1) Performance supposed to be possessed by the glass material is not completely evaluated by the conventional technique. Namely, the glass material has a characteristic that does not appear by a testing and evaluating method of the conventional technique.
(Reason 2) Processing which is not performed in testing and evaluating the glass material, has an influence on the actual surface of the lens as an external factor.

First, the reason 1 is as follows. Each type of chemical durability including acid resistance, etc., is evaluated by the conventional technique, and the conventional technique is pursuant to the content defined in the Japan Optical Glass Industry Association Standard. Therefore, hydrogen ion concentration index (called “pH” hereafter) in a process liquid of each type of index to be evaluated in which a glass material sample is immersed, is fixedly defined. Further, evaluation conditions are not equalized in each type of indexes, regarding not only pH of the process liquid, but also a shape and a size of the glass material, immersion time required for immersion into the process liquid, and a liquid temperature, etc.

Further, the conventional technique employs discrete evaluation stages such as grade 1 to grade 6, and does not employ a more detailed technique of classifying and evaluating the glass material having more excellent durability than grade 1.

From these facts, the inventors of the present invention consider as follows: the point is that an influence of pH variation of the process liquid in which the glass material is immersed, cannot be grasped by the evaluation of the conventional technique, and it appears that this point shows that the present invention has a characteristic that is not grasped by the conventional method for testing and evaluating the glass material.

Next, the reason 2 is as follows. A producing step of fabricating a lens generally includes a cleaning step using a cleaning liquid. Then, in the cleaning step, a cleaning power is improved by using strongly alkaline cleaning liquid. Probably the inventors of the present invention assume that pH of the cleaning liquid used in the cleaning step has an influence on the state of the lens surface as the external factor, thus degrading the state of the lens surface with elapse of time.

As a result of strenuous efforts by the inventors of the present invention based on the above-described reasons 1 and 2, irrespective of a common sense that the contents defined by the Japan Optical Glass Industry Association Standard should be complied with, a completely new unconventional concept is found as follows. Namely, pH of the process liquid is varied and the influence thereof is tested and evaluated. Thus, based on such a concept from a different angle, the inventors of the present invention obtains a knowledge that the above-described problems can be solved by performing test and evaluation so that the influence of pH on the chemical durability of the glass material (namely the characteristic that is not grasped by the test and the evaluation based on the conventional technique) can be grasped, separately from the contents defined by the Japan Optical Glass Industry Association Standard.

Then, the aforementioned knowledge is put into practice, and a correlation between pH and the chemical durability is grasped and by utilizing such a correlation, the inventors of the present invention obtain a knowledge that the generation of the gap between the evaluation applied to the glass material and the actual surface state of the glass material can be prevented even after the glass material is actually turned into a product such as an optical element, which is the glass material whose chemical durability is evaluated to be a high grade by the conventional technique.

In addition, even in a case of the glass material whose chemical durability is evaluated to be a low grade by the conventional technique, by setting pH of the process liquid used for producing the optical element in a range of the pH determined for each type of the glass material, the chemical durability of the glass material can be sufficiently extracted in some cases, so as not to be inferior to the optical element based on the glass material whose chemical durability is evaluated to be a high grade.

Based on such a new knowledge by the inventors of the present invention, the present invention is provided, and a first aspect of the present invention provides a method for testing a glass material, to perform a test regarding a chemical durability of a glass material, comprising:

examining a correlation between a variation of hydrogen ion concentration indexes of a process liquid in which the glass material is immersed, and a variation of chemical durability index values of a prescribed type of the glass material.

A second aspect of the present invention provides the method for testing a glass material according to the first aspect, wherein haze values of the glass material after being immersed in the process liquid for a prescribed time, are used as the chemical durability index values.

A third aspect of the present invention provides the method for testing a glass material according to the first or second aspect, wherein weight variation values of the glass material before/after being immersed in the process liquid for a prescribed time, are used as the chemical durability index values.

A fourth aspect of the present invention provides the method for testing a glass material, wherein based on a correlation in each type of the glass materials examined by the method for testing a glass material according to any one of the first to third aspects, a hydrogen ion concentration index of the process liquid used for processing each type of the glass material, is selected, so that corresponding chemical durability index values are set in an allowable range.

A fifth aspect of the present invention provides a method for producing an optical element, comprising:

testing a glass material in each type, to examine a correlation between a variation of hydrogen ion concentration indexes of a process liquid in which a glass material is immersed, and a variation of chemical durability index values of a prescribed type of the glass material;

evaluating the glass material by selecting a hydrogen ion concentration index of the process liquid to be used for processing the glass material, so that a corresponding chemical durability index value is set in an allowable range, based on a correlation in each type of the glass materials examined in the step of testing the glass material; and

processing the glass material using the process liquid having the hydrogen ion concentration index selected in the step of evaluating the glass material, to thereby produce an optical element.

According to the present invention, the influence of the variation of the hydrogen ion concentration indexes on the chemical durability index values, can be grasped. Therefore, the generation of the gap between the evaluation for the glass material, and the actual surface state of the glass material can be prevented. As a result, the chemical durability possessed by the optical element can be sufficiently exhibited.

### BRIEF DESCRIPTION OF THE DRAWINGS

FIG. 1 is a view in which Abbe number and a refractive index possessed by each type of glass materials are plotted by center points of hexagons indicating each type of the glass materials, in which the Abbe number vd is taken on the horizontal axis, and the refractive index nd is taken on the vertical axis. Note that a chemical durability index based on the Japan Optical Glass Industry Association Standard JOGIS07 is indicated by a region at each side of each hexagon showing each type of the glass materials. Also, note that the type of the chemical durability index is different depending on the region at each side as follows: an upper side region of the hexagon indicates a water resistance class powder method (Dw), and a region adjacent to the upper side region of the hexagon clockwise therefrom (namely upper right region) indicates an acid resistance class powder method (D_{A}), and a lower right region indicates a blue fogging resistance surface method (T_{blue}) r and a lower side region of the hexagon indicates a latent flow resistance (D_{NaOH}), and a lower left region indicates a latent flaw resistance (D_{STTP}), and an upper left region indicates a chemical durability (D_{O}). Further, a difference in grades of the chemical durability is shown by a pattern in each region.

FIG. 2A is a view showing a result of a test performed to a glass material of example 1 (glass type: FDS18), in which pH is taken on the horizontal axis, haze values (%) are taken on a right vertical axis, and weight variation values (g) are taken on a left vertical axis, which are plotted in this figure. FIG. 2B is a view showing a result of a test performed to a glass material of example 16 (glass type: M-FCD1), in which pH is taken on the horizontal axis, haze values (%) are taken on the right vertical axis, and weight variation values (g) are taken on the left vertical axis, which are plotted in this figure.

### DESCRIPTION OF DETAILED EMBODIMENT OF THE INVENTION

Detailed embodiments of the present invention will be described hereafter.

This embodiment will be described in detail in the following order.

1. A method for testing a glass material
A) Preparation of a glass material
B) Preparation of a process liquid
C) Setting of a chemical durability index value (haze value/weight variation value)
   2. A method for evaluating a glass material
   3. A method for producing an optical element
A) Step of testing a glass material
B) Step of evaluating a glass material
C) Step of processing a glass material
   4. Effect of this embodiment
   5. Modified example

### 1. A Method for Testing a Glass Material>

### A) Preparation of a Glass Material

First, a glass material for a test is prepared for each type of the glass material being an object to examine the chemical durability. Such a glass material is also called “a glass sample” hereafter.

Note that the glass material mentioned here includes a material for forming an optical element (an optical glass), wherein a chemical composition is different depending on a type of the glass material (simply called a “glass type” hereafter). Accordingly, the chemical durability of the glass material is also different depending on a different glass type.

For example, the glass sample is processed as follows. Namely, the glass material is processed into a disc shape in a diameter of 43.7 mm and a thickness of 5 mm, and a sand-falling test is performed to two main opposed surfaces (planes with a diameter of 43.7 mm) with granularity of #1200 defined in JIS R 6001 (granularity of a polishing material), using A-abrasive grain defined in JIS R 6111 (artificial abrasive). Then, the glass material thus processed is polished using a straight asphalt pitch and cerium oxide (CeO₂) to be finished so that a grey is not observed through a loupe, to thereby obtain the glass sample. However, the glass sample is not limited thereto, and other one may be acceptable, provided that it can be immersed in the process liquid.

### B) Preparation of a Process Liquid

Further, in addition to the glass sample, process liquid is prepared, for immersing the glass sample in a container having a size capable of containing the glass sample. In this case, the process liquid allowing the hydrogen ion concentration index (pH) to be suitably adjusted, is prepared. It can be considered that pH adjustment is performed in such a way that pure water is used as neutral process liquid, and nitric acid (HNO₃) is added when the pure water is turned into acidic, and sodium hydroxide (NaOH) is added when the pure water is turned into alkaline. Namely, HNO₃ and NaOH are used for the pure water, and a mixing ratio of the HNO₃ and NaOH is varied. This can be given as a specific example of the process liquid. Note that the process liquid is not limited thereto, and other process liquid may also be used provided that the pH adjustment can be performed.

After the glass sample and the process liquid are prepared, the glass sample is immersed in the process liquid which is adjusted to a certain pH and maintained to have a prescribed temperature (for example 50° C.) for a prescribed time (for example 15 hours). Note that the temperature and the time, etc., may be suitably set and are not limited to specific values.

Note that liquid (such as polishing liquid and cleaning liquid) used in the step of processing the glass material in the <3. A method for producing an optical element> is assumed to be used as the process liquid. Namely, the liquid used until completion of the optical element as a product is used as the process liquid. Then, the glass sample is immersed in this process liquid, and a variation manner of the chemical durability index values according to the pH variation, is tested. Then, based on a test result thereof, the glass material is evaluated for each type of the glass material, and a pH range of the process liquid is determined, which is the process liquid used in the step of processing the glass material when the optical element is produced. Then, in an actual step of processing the glass material, the process liquid (such as polishing liquid and cleaning liquid) in this pH range is used. Namely, the process liquid used in <1. A method for testing a glass material> is required to be used as the polishing liquid and the cleaning liquid which are used in the step of processing the glass material for producing the optical element.

### C) Setting of the Chemical Durability Index Values (Haze Values and Weight Variation Values)

Thereafter, the chemical durability of the glass material is tested. More specifically, prescribed kinds of chemical durability index values are measured, which are the chemical durability index values for the samples of the glass material extracted from the process liquid. Based on this measurement, the correlation between the pH variation of the process liquid in which the glass sample is immersed, and a variation of the prescribed kinds of chemical durability index values in the glass sample immersed in the process liquid, is examined for each type of the glass sample.

The chemical durability of the glass material means the durability when a chemical reaction occurs between a component of the glass material and the process liquid (such as polishing liquid and cleaning liquid), and the chemical durability index values are used as an objective reference (index) for evaluating such a durability.

Various kinds of index values can be used as the chemical durability index values, and the “haze values” and the “weight variation values” are used as the prescribed kinds of chemical durability index values in this embodiment. This is because by using the “haze values” and the “weight variation values”, and particularly using the “haze values”, the degradation of a surface state of the glass sample with elapse of time (for example the degradation in the state of the glass material due to fogging) can be precisely and objectively grasped.

“Haze value” is a value indicating a degree of a so-called fogging, showing that the smaller the value is, the higher the transparency is. More specifically, the haze value is expressed by the following equation: Haze value (%)=T_{d}/Tₜ×100 (T_{d}: diffusion transmittance, Tₜ: total light transmittance). Such a haze value can be measured using a haze meter defined in “Method (surface method) for measuring chemical durability of optical glass 07-1975 by Japan Optical Glass Industry Association Standard JOGIS”, and allowing the measured light to be transmitted vertically to two opposed main surfaces of the glass sample after being immersed in the process liquid for a prescribed time. Note that in the specification of the present application, the haze value has a similar meaning as “haze” in the “Method (surface method) for measuring chemical durability of optical glass 07-1975 by Japan Optical Glass Industry Association Standard JOGIS”.

“Weight variation value” is the value indicating a weight variation (reduction amount) of the glass sample before/after immersion into the process liquid. Such a weight variation value (g) can be measured by measuring a weight of the glass sample before/after immersion into the process liquid for a prescribed time, and calculating a differential value obtained by each measurement.

After measuring the haze value and the weight variation value of the glass sample immersed in the process liquid which is adjusted to a certain pH for a prescribed time, the haze value and the weight variation value of the sample of a new glass sample of the same type of glass as the aforementioned glass sample, is measured using the process liquid with pH changed, under the same condition of the process liquid excluding pH. Similar measurement technique is used as the measurement technique used before changing the pH. Namely, the haze value and the weight variation value are measured again only by changing the pH of the process liquid.

Thus, the haze value and the weight variation value corresponding to each of at least two or more pH can be obtained. Therefore, the correlation between the variation of the pH of the process liquid and the variation of the haze values in the samples of the glass material, and the correlation between the variation of the pH of the process liquid and the variation of the weight variation values in the samples of the glass materials, can be grasped in each type of the glass samples. Namely, information regarding the correlation between the variation of pH and the variation of the chemical durability index values (more specifically the variation of the haze values and the weight variation values), can be obtained in each type of the glass material, by testing the glass materials using the aforementioned methods.

Note that the haze value and the weight variation value may be measured, regarding at least two pH. This is because the correlation between the variation of pH and the variation of the chemical durability index values can be grasped by performing the measurement regarding at least two pH. However, preferably three or more pH from acidity to alkalinity should be measured, like pH=3, 2, 6.3, 9, 9.4, 11.8. This is because precision of specifying the correlation can be improved by improving a resolving power, when three or more pH are measured.

### 2. A Method for Evaluating a Glass Material

In this embodiment, based on the correlation in each type of the glass materials examined by the aforementioned method for testing a glass material, pH of the process liquid used for processing each type of the glass material is selected so that corresponding chemical durability index value is set in an allowable range. This allowable value can be arbitrarily determined by a person who processes the glass material.

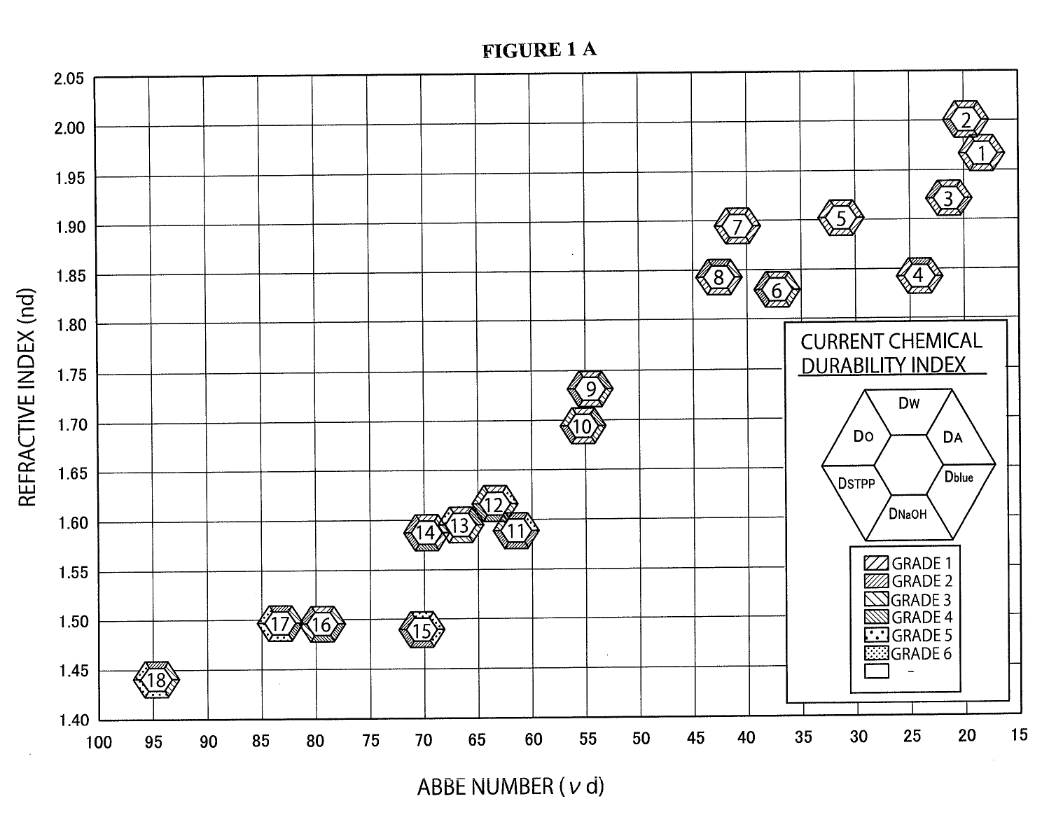

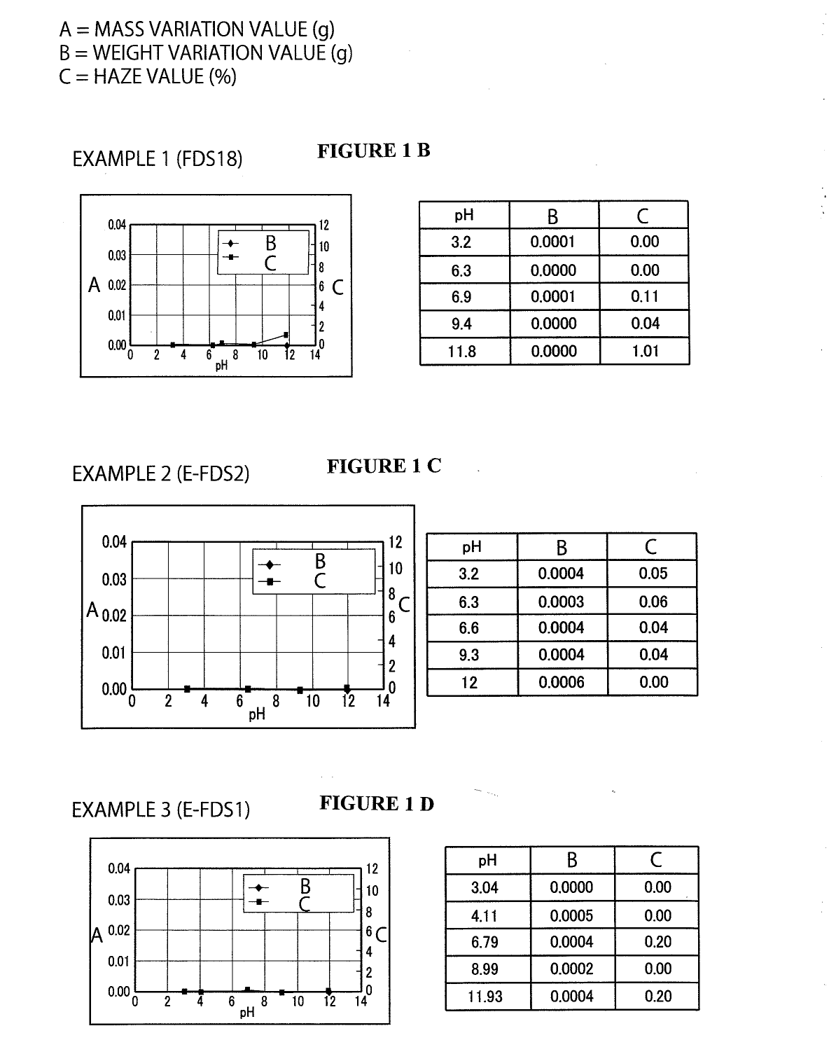

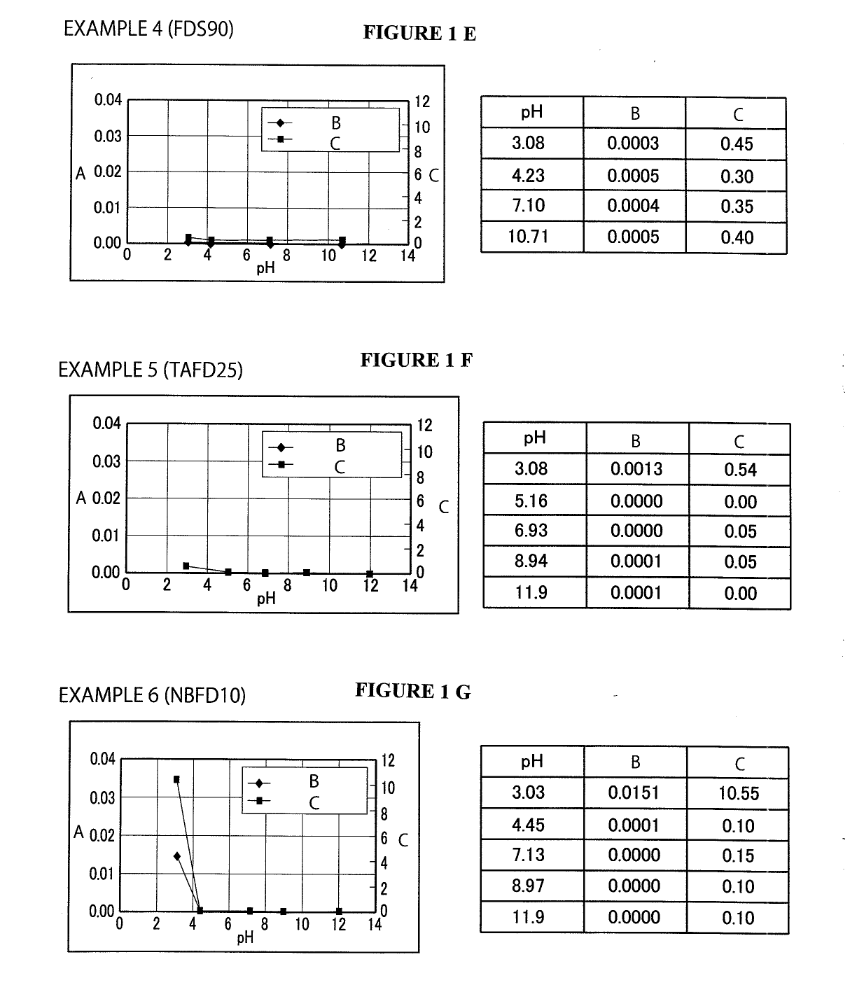

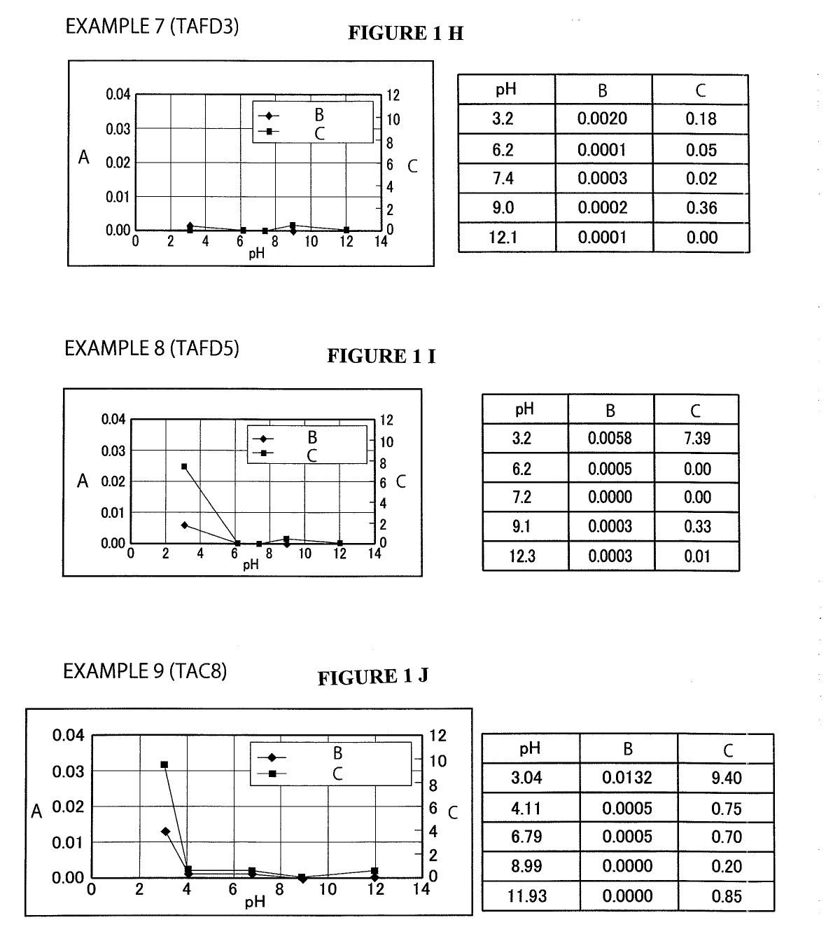

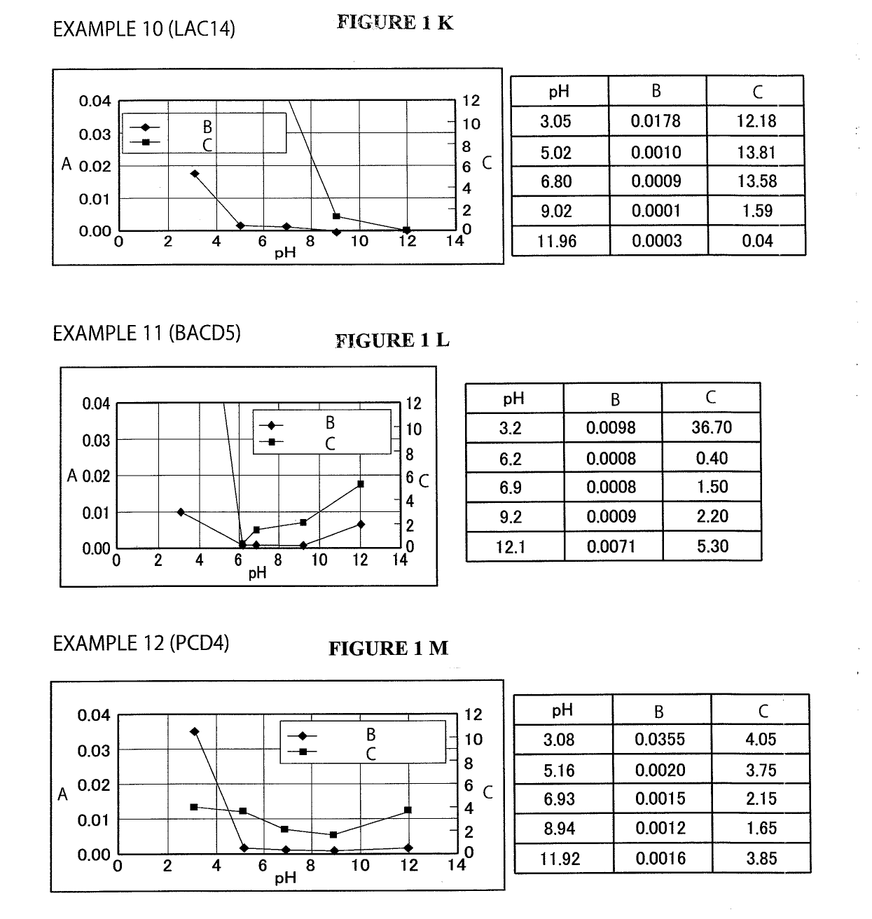

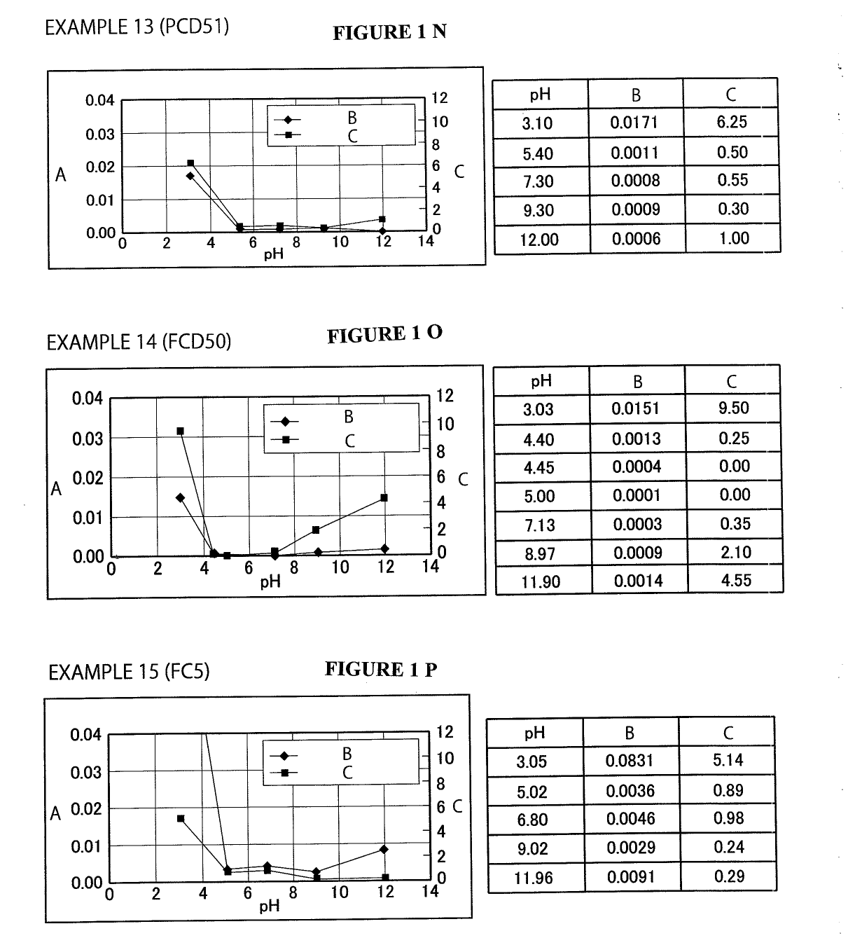

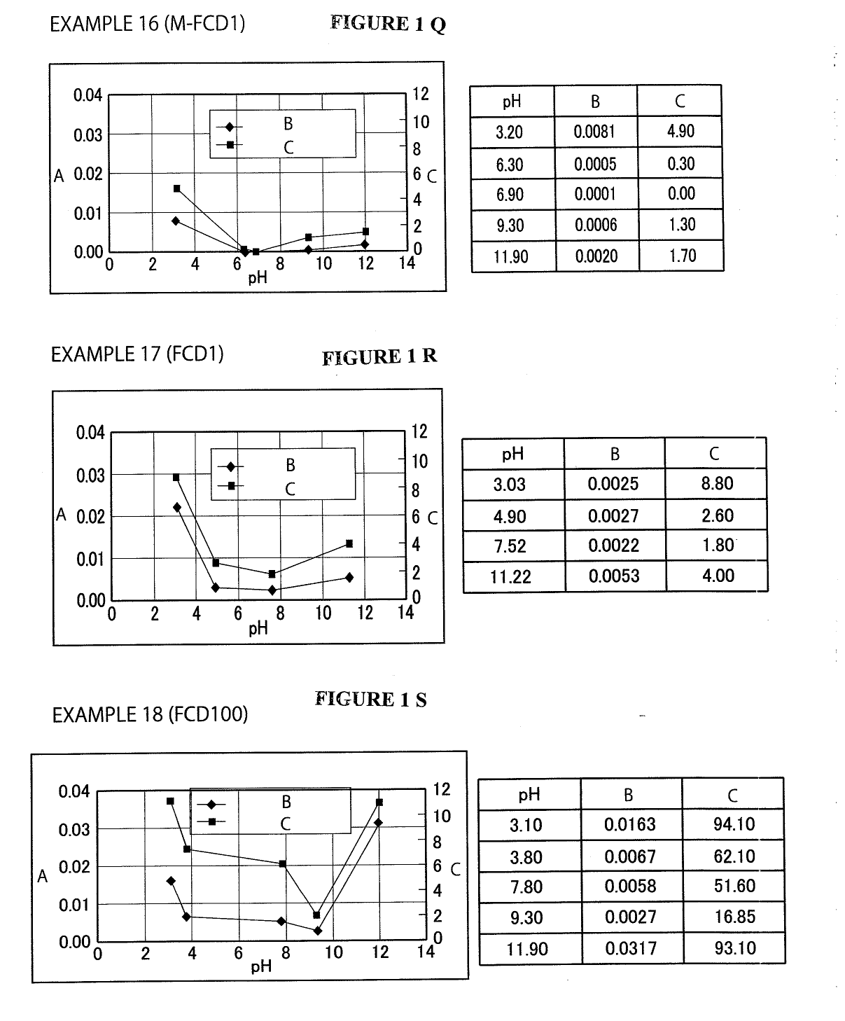

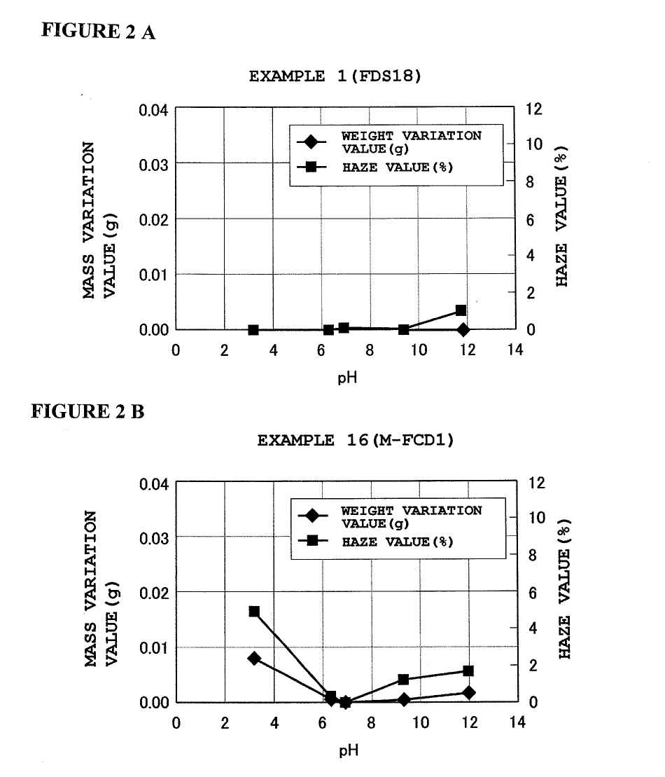

A result obtained by the method for testing the glass material will be described hereafter, and FIG. 1 and FIG. 2 are used for evaluating the glass material using this result.

FIG. 1 is a view in which Abbe number vd and a refractive index nd possessed by each type of glass materials are plotted by center points of hexagons indicating each type of the glass materials. Note that a chemical durability index based on the Japan Optical Glass Industry Association Standard JOGIS07 is indicated by a region at each side of each hexagon showing each type of the glass materials. Also, note that the kind of the chemical durability index is different depending on the region at each side as follows: an upper side region of the hexagon indicates a water resistance class powder method (Dw), and a region adjacent to the upper side region of the hexagon clockwise therefrom (namely upper right region) indicates an acid resistance class powder method (D_{A}), and a lower right region indicates a blue fogging resistance surface method (T_{blue}), and a lower side region of the hexagon indicates a latent flow resistance (D_{NaoH}), and a lower left region indicates a latent flaw resistance (D_{STTP}), and an upper left region indicates a chemical durability (D_{O}). Further, a difference in grades of the chemical durability is shown by a pattern in each region. Note that “−” described in a display of the grade shows that due to elution of an overall surface of the glass material when being immersed into the process liquid, a blue fogging layer is not observed or the variation of an interference color is irregular, thus making it impossible to perform evaluation based the grade.

FIG. 2A is a view showing a result of a test performed to a glass material of example 1 (glass type: FDS18) as will be described later, in which pH is taken on the horizontal axis, haze values (%) are taken on a right vertical axis, and weight variation values (g) are taken on a left vertical axis, which are plotted in this figure. FIG. 2B is a view showing a result of a test performed to a glass material of example 16 (glass type: M-FCD1) as will be described later, in which pH is taken on the horizontal axis, haze values (%) are taken on the right vertical axis, and weight variation values (g) are taken on the left vertical axis, which are plotted in this figure.

For example, when the glass material of example 1 as will be described later (glass type: FDS18) is used, as shown in FIG. 2A, pH of the process liquid can be set in a range of at least 3.2 or more and 11.8 or less by using the haze value as the chemical durability index value and setting its allowable value to 2% or less. Further, pH of the process liquid is set in the same range even if the weight variation value is used as the chemical durability index value and its allowable value is set to 0.01% or less.

Meanwhile, when the allowable value of the haze value is set in the vicinity of 0% (for example, 0.20% or less), it is found that pH of the process liquid is required to be set in a range of at least 3.2 or more and 9.4 or less.

Namely, as shown in FIG. 1, in the glass material having the glass type of FDS18, the grade of the chemical durability of the conventional technique is high entirely, and meanwhile the polishing liquid and the cleaning liquid are required to be set in the aforementioned pH range at the time of producing the optical element, and this can be grasped before producing the optical element.

Further, as another sample, the glass material (glass type: M-FCD1) of example 16 is used as will be describe later, and this case is given as another example. When this glass material is tested by the conventional technique, as shown in FIG. 1, the grade of the chemical durability is entirely low. Regarding this glass material, the correlation between the variation of the pH values and the variation of the haze value can be obtained as shown in FIG. 2B by using the method for testing the glass material of this embodiment. According to this figure, the haze value of 1% or less can be achieved by setting the pH of the process liquid in a range of 6.3 or more and 6.9 or less. Namely, although this glass material is relatively inferior to the glass material of example 1 (glass type: FDS18) in terms of the grade of the chemical durability of the conventional technique, the optical element having high chemical durability (low haze value here) which is not inferior to a high grade glass material, can be obtained by setting the pH of the process liquid to 6.3 or more and 6.9 or less, which is the process liquid used for producing the optical element as a product. In addition, by using the method for testing a glass material and the method for evaluating a glass material of this embodiment, the above-described low haze value can be realized as predicted initially, even after the optical element is turned into a product.

Generally, when the glass material with low refractive index and high Abbe number is used, as shown in FIG. 1, it is inevitable that the grade of the chemical durability of the conventional technique is lowered. However, even in a case of using the glass material having low refractive index and high Abbe number by using the method for testing a glass material and the method for evaluating a glass material according to this embodiment, the pH range of the process liquid according to the glass type can be obtained by the method for testing a glass material. Then, the chemical durability corresponding to that of the glass material having high refractive index and low Abbe number can be obtained by using the process liquid having this pH range when the glass material is processed.

Namely, even in a case that the glass material has any kind of refractive index and Abbe number, the chemical durability which is supposed to be originally possessed by the glass material, namely a potential chemical durability which is not grasped by the conventional technique, can be exhibited.

As a result, even in a case of the glass material which is refrained from being used because the person who processes the glass material receives a low grade evaluation regarding the chemical durability, such a glass material can be freely selected. Then, the optical element capable of exhibiting sufficient chemical durability can be produced, while having a desired refractive index and Abbe number.

### 3. A Method for Producing an Optical Element

The method for producing an optical element will be described hereafter.

Although the optical element using the glass material can be utilized, an optical glass lens (simply called a lens) is produced in this embodiment, and this case will be described hereafter.

In this embodiment, the step of testing a glass material using the aforementioned method for testing a glass material, and the step of evaluating a glass material using the aforementioned method for evaluating a glass material, are performed to the glass test sample. After a suitable pH range of the process liquid is obtained in both steps, the step of processing a glass material is performed to the glass material being the base of the optical element as a product, using the process liquid with limited pH range.

Each step will be described hereafter, and a portion overlapped with already descried contents is omitted.

### A) The Step of Testing a Glass Material

First, the glass type in the lens is selected. In this embodiment, the same glass type as that of example as will be described later (glass type: FDS18) is selected, and the step of testing a glass material and the step of evaluating a glass material are performed to the glass material of this glass type. Further, the haze value and the weight variation value are used for the chemical durability index values in the step of testing a glass material. As a result, as shown in FIG. 2A, the correlation between the haze value and the weight variation value regarding the variation of pH of the process liquid can be obtained in the step of testing a glass material.

### B) The Step of Evaluating a Glass Material

Then, the allowable value in the step of evaluating a glass material is set so that the haze value is 1% or less and the weight variation value is set to 0.001 g or less. By this setting, the pH range of the liquid used in the step of processing a glass material is determined to 3.2 or more and 9.8 or less.

### C) The Step of Processing a Glass Material

Based on the result obtained by the step of evaluating a glass material, the step of processing a glass material is performed to the glass material being a base of the lens as a product, which is the same glass material as the glass type selected in the step of testing a glass material and the step of evaluating a glass material. More specifically, the step of processing a glass material includes a polishing step and a cleaning step performed to the lens. pH of the polishing liquid in the polishing step is set in a pH range selected in the step of evaluating a glass material described above. Similarly, pH of the cleaning liquid in the cleaning step is set in a pH range selected in the step of evaluating a glass material described above. Namely, the step of processing a glass material includes the step of producing the optical element by processing the glass material using the process liquid having pH selected in the step of evaluating a glass material described above.

Note that a publicly-known method may be used as the method for fabricating the lens. For example, as a specific step such as melting of a glass source in the lens, cast, pipe flow, roll, and press, which are conventionally known method, may be used. In addition, the publicly-known method may also be used as the step required for producing an optical glass lens, being the step not described in this specification. The publicly-known method includes U.S. Pat. No. 4,747,039, U.S. Pat. No. 4,726,666, and U.S. Pat. No. 3,361,270, etc., by the present applicant.

### 4. Effect of this Embodiment

According to this embodiment, the following effect is exhibited.

The correlation between pH and the chemical durability is grasped and utilized. Therefore, in the glass material with the chemical durability by the conventional technique evaluated to be a high grade, the generation of the gap between the evaluation given to the glass material and the actual surface state of the glass material can be prevented, even after the glass material is turned into a product actually.

In addition, even in a case of the glass material with the chemical durability evaluated to be a low grade by the conventional technique, pH of the process liquid used for producing the optical element is set in a range determined for each type of the glass material. Therefore, the chemical durability of the glass material can be sufficiently extracted in some cases, so as not to be inferior to the optical element based on the glass material with the chemical durability evaluated to be a high grade.

Namely, even in a case that the glass material has any kind of refractive index and Abbe number, the chemical durability supposed to be possessed by the glass material originally, which is a potential chemical durability not grasped by the conventional technique can be exhibited.

As a result, even in a case of the glass material which is refrained from being used because the person who processes the glass material receives a low grade evaluation regarding the chemical durability, such a glass material can be freely selected. Then, the optical element capable of exhibiting sufficient chemical durability can be produced, while having a desired refractive index and Abbe number.

Owing to the above-described effects, there is provided the method for producing the optical element for the purpose of providing the method for testing a glass material and the method for evaluating a glass material capable of preventing the gap between the evaluation result and the actual surface state of the lens, and as a result, capable of sufficiently exhibiting the chemical durability possessed by the optical element.

### 5. Modified Example

### (When the Chemical Durability Index Value Includes the Haze Value Only or the Weight Variation Value Only)

This embodiment describes a case that the chemical durability index value includes two values (namely the “haze value” and the “weight variation value”). Meanwhile, the concept of the present invention can also be applied to a case that the chemical durability index value is one. More specifically, in the method for testing a glass material and the method for evaluating a glass material according to this embodiment, the correlation between the variation of pH of the process liquid, and the haze value is grasped, and from this correlation, a suitable pH range may be obtained.

Reversely to the above-described case, the concept of the present invention can also be applied to a case that the chemical durability index value includes the weight variation value only.

### (Other Chemical Durability Index Value (Surface Degradation))

As described above, various kinds of index values can be used as the chemical durability index value, other than the “haze value” and the “weight variation value”, provided that it shows chemical durability. As an example thereof, “degradation of the surface state” can be given. As shown in table 1 of example 1 (glass type: FDS18) as will be described later, predicted pH range of the process liquid used in the step of processing a glass material may be determined by examining the correlation between the variation of pH of the process liquid and the degradation of the surface state of the glass material. For example, as shown in table 1 of example 1, the pH range is preferably set to at least 3.2 or more and 9.4 or less in a case that the glass type is FDS18.

### (Optical Element)

This embodiment describes a case that the optical element is the optical glass lens. As a specific example of this optical element, each kind of lens such as a spherical lens, an aspherical lens, a micro lens, diffraction grating, a lens with diffraction grating, a lens array, and a prism, etc., can be given. Further, from a surface shape, a concave meniscus lens, a biconcave lens, a plano-concave lens, a convex meniscus lens, a biconvex lens, and a plano-convex lens, etc., can be given.

Note that these lenses can be optical elements by providing thereon an optical thin film such as an antireflection film, a total reflection film, a partial reflection film, and a film having spectral characteristics as needed.

Further, the aforementioned optical element is suitable as a component of a compact image pick-up optical system with high performance, and is suitable for the image pick-up optical system such as a digital still camera, a digital video camera, a camera mounted on a cell phone, and a camera mounted on a vehicle.

### EXAMPLES

The present invention will be more specifically described next, with reference to examples. It is a matter of course that the present invention is not limited to the following examples.

### Example 1

### (The Step of Testing a Glass Material)

First, the step of testing a glass material is performed. The glass material of the glass type FDS18 (by HOYA Corporation) was used in this example, as the glass sample used in this test. Then, similarly to the example given in this example, the glass sample was tested as follows. The glass material was processed into a disc shape with a diameter of 43.7 mm and a thickness of 5 mm, and a sand-falling test was performed to two main opposed planes (planes with a diameter of 43.7 mm) with granularity of #1200 defined in JIS R 6001 (granularity of a polishing material), using A-abrasive grain defined in JIS R 6111 (artificial abrasive). Then, the glass material thus processed was polished using a straight asphalt pitch and cerium oxide (CeO₂) to be finished so that a grey is not observed through a loupe, to thereby fabricate the glass test sample into a lens shape, which is a test target in the step of testing a glass material.

Further, 5 kinds of pH=3.2, 6.3, 6.9, 9.4, 11.8 were prepared as the process liquid. Note that the process liquid of pH=3.2 was prepared by diluting nitric acid (HNO₃) having a concentration of 6.3% with pure water to thereby adjust pH. Pure water was used as the process liquid of pH=6.3. The process liquid of pH=6.9, 9.4, 11.8 was prepared by diluting sodium hydroxide (NaOH) having a concentration of 0.4% with pure water, to thereby adjust the pH. Note that when the liquid with pH=7 or around was measured by a pH meter, it was difficult to perform measurement by fluctuation of measured values. Therefore, pH was measured by using pH litmus paper in which a fluctuation range of the measured values is narrow.

Meanwhile, the following apparatus was used as the apparatus for performing this test. First, a water bath was prepared. Then, two plastic containers disposed in the water bath were prepared. A plastic glass material sample enclosing container was provided in each plastic container for putting therein the glass test sample. Note that a holder for holding the glass test sample, is provided in the glass material sample enclosing container.

Then, two samples of the glass material for testing, being a test object, are disposed in each glass material sample enclosing container. The process liquid (500 ml) with two test samples of the glass material immersed therein, was enclosed in one of the glass material sample enclosing container. Then, the same amount of aqueous RO was enclosed in the other glass material sample enclosing container instead of the process liquid. In addition, the glass test sample immersed in the aqueous RO was used for measuring the haze value of the glass test sample immersed in the process liquid, which was then used for sensitivity correction at the time of measuring the haze value by a haze meter.

Thereafter, aqueous RO (800 ml) was enclosed in each plastic container, in a state of inserting each glass sample enclosing container. Thus, a water level in the plastic container is set to be higher than the water level of the process liquid or the aqueous RO in the glass sample enclosing container.

Then, two plastic containers were disposed in the water bath, wherein the water bath was filled with distilled water and a temperature was adjusted to about 50° C. At this time, the water level in the water bath was set to be higher than the water level of the aqueous water RO in the plastic container. The time required for immersion into the process liquid and the aqueous RO was set to 15 hours. After immersion into the process liquid, the haze value, the weight variation value, and a surface degradation state of the glass test sample were measured.

Then, such a measurement was performed in each prepared process liquid (5 kinds of pH).

As described above, the correlation between the pH variation and the chemical durability index values (haze values, weight variation values, and surface degradation state) was grasped in example 1. Results thereof were shown in example 1 of FIG. 1, and table 1 as described below.

Note that table 1 is a table indicating the correlation between the pH values of the process liquid and the surface degradation state.

TABLE 1

| | Surface degradation |
|---|---|
| pH | state |
| 3.2 | No variation |
| 6.3 | No variation |
| 6.9 | No variation |
| 9.4 | No variation |
| 11.8 | Fine flaw on the |
| | surface |

Further, FIG. 2A shows an expanded result of example 1.

### (Step of Evaluating Glass Material)

pH of the process liquid used for processing each type of the glass material was selected based on the correlation in each type of the glass materials examined in the step of testing a glass material, so that a corresponding chemical durability index value was set in an allowable range. Namely, in this example, the value in a range of the haze value of 1% or less, and in a range of the weight variation value of 0.01 g or less was set as the allowable value. Further, in the surface degradation state shown in table 1 as well, the state of not allowing the fine flaw to be formed on the surface, was set in a range of the allowable value. Thus, pH range of the cleaning liquid used for producing the product was set to 3.2 or more and 9.4 or less.

### (Step of Processing a Glass Material)

After the pH range of the polishing liquid and the cleaning liquid was determined by the step of evaluating a glass material, the glass sample for product use being the same kind of glass material as the glass test sample, was cleaned by the cleaning liquid in which pH was set in the aforementioned range. Then, this glass sample for product use was left to stand for one day at room temperature.

### (Evaluation)

When the surface of the glass sample for product use was observed, a latent flaw was not generated on the surface of the glass sample for product use, and the degradation state of the glass sample for product use with elapse of time was not observed. Namely, the glass sample for product use actually having excellent chemical durability comparable to the evaluation of the chemical durability by the conventional technique, could be obtained.

### Examples 2 to 18

### (Step of Testing a Glass Material and Step of Evaluating a Glass Material)

Similarly to example 1, the step of testing a glass material and the step of evaluating a glass material were performed to the glass test sample of the glass type selected in each example. Then, as shown in FIG. 1, results of a plurality of types of the glass material that exist on a glass map classified by refractive index nd and Abbe number vd, were listed in tabulation on the glass map individually for each glass type.

Note that the tabulation called here shows the information to be provided, namely the correlation between the variation of pH and the variation of the chemical durability index values, which are indicated by a figure (graph) or table, or both of them. Further, “ . . . individually for each type of the glass material,” means that the results are shown separately for each glass type one by one.

### (Step of Processing a Glass Material)

Similarly to example 1, the pH range of the polishing liquid and the cleaning liquid were determined by the step of evaluating a glass material, and thereafter, cleaning was performed to the glass sample for product use being the same type of glass material as the glass test sample. Then, the glass sample for product use was left to stand at room temperature for 1 day.

### (Evaluation)

When the surface of the glass sample for product use was observed, similarly to example 1, the latent flow was not generated on the surface of the glass sample for product use, and the degradation state of the glass sample for product use with elapse of time, was not observed. Namely, the glass sample for product use actually having excellent chemical durability comparable to the evaluation of the chemical durability by the conventional technique, could be obtained.

Further, when the glass material of example 16 (glass type: M-FCD1) was used, the glass sample for product use having high chemical durability not inferior to a high grade glass material, could be obtained by setting pH of the process liquid used in the step of processing a glass material to 6.3 or more and 6.9 or less, irrespective of a matter that the glass material of example 16 is relatively inferior to the glass material of example 1 (glass type: FDS18) in terms of the grade of the chemical durability of the conventional technique.

1. A method for testing a glass material, to perform a test regarding a chemical durability of a glass material, comprising:
examining a correlation between a variation of hydrogen ion concentration indexes of a process liquid in which the glass material is immersed, and a variation of chemical durability index values of a prescribed type of the glass material.

2. The method for testing a glass material according to claim 1, wherein haze values of the glass material after being immersed in the process liquid for a prescribed time, are used as the chemical durability index values.

3. The method for testing a glass material according to claim 1, wherein weight variation values of the glass material before/after being immersed in the process liquid for a prescribed time, are used as the chemical durability index values.

4. A method for evaluating a glass material, wherein based on a correlation in each type of the glass materials examined by the method for testing a glass material according to claim 1, a hydrogen ion concentration index of the process liquid used for processing each type of the glass material, is selected, so that corresponding chemical durability index values are set in an allowable range.

5. A method for evaluating a glass material, wherein based on the correlation in each type of glass materials examined by the method for testing a glass material according to claim 3, a hydrogen ion concentration index of the process liquid used for processing each type of the glass material is selected, so that corresponding chemical durability index values are set in an allowable range.

6. A method for producing an optical element, comprising:
testing a glass material in each type, to examine a correlation between a variation of hydrogen ion concentration indexes of a process liquid in which a glass material is immersed, and a variation of chemical durability index values of a prescribed type of the glass material;
evaluating the glass material by selecting a hydrogen ion concentration index of the process liquid to be used for processing the glass material, so that a corresponding chemical durability index value is set in an allowable range, based on a correlation in each type of the glass materials examined in the step of testing the glass material; and
processing the glass material using the process liquid having the hydrogen ion concentration index selected in the step of evaluating the glass material, to thereby produce an optical element.

7. The method for testing a glass material according to claim 2, wherein weight variation values of the glass material before/after being immersed in the process liquid for a prescribed time, are used as the chemical durability index values.

8. A method for evaluating a glass material, wherein based on the correlation in each type of glass materials examined by the method for testing a glass material according to claim 7, a hydrogen ion concentration index of the process liquid used for processing each type of the glass material is selected, so that corresponding chemical durability index values are set in an allowable range.

9. A method for evaluating a glass material, wherein based on a correlation in each type of the glass materials examined by the method for testing a glass material according to claim 2, a hydrogen ion concentration index of the process liquid used for processing each type of the glass material, is selected, so that corresponding chemical durability index values are set in an allowable range.
